(19) United States
(12) Patent Application Publication
(10) Pub. No.: US 2003/0138407 A1
(43) Pub. Date: Jul. 24, 2003

(54) Therapeutic methods for nucleic acid delivery vehicles

(76) Inventors: Patrick Lu, Gaithersburg, MD (US); Puthupparampil Scaria, Montgomery Village, MD (US); Martin C. Woodle, Bethesda, MD (US); Frank Xie, Rockville, MD (US); Jun Xu, Rockville, MD (US); Quinn Tang, Rockville, MD (US)

Correspondence Address: HELLER EHRMAN WHITE & MCAULIFFE LLP, 1666 K STREET,NW, SUITE 300, WASHINGTON, DC, 20006, US

(21) Appl. No.: 10/286,956

(22) Filed: Nov. 4, 2002

Related U.S. Application Data
(60) Provisional application No. 60/330,909, filed on Nov. 2, 2001.

(51) Int. Cl. A61K 48/00; C12N 15/86

(52) U.S. Cl. 424/93.2; 514/44; 435/456

(57) ABSTRACT

It has been found that certain synthetic vectors and nucleic acid sequences that encode viral genomic sequences can, for example, be administered to a subject repeatedly as a vehicle for effectively delivering one or more therapeutic nucleic acid molecules or polypeptides to a cell or tissue. Accordingly, the disclosed nucleic acid delivery vehicles can be used, for instance, as part of a therapeutic regimen that involves an ongoing use of a therapeutic nucleic acid molecule or polypeptide.

### FIELD OF THE INVENTION

The invention relates to methods of delivering one or more therapeutic compositions to a cell or a tissue in a mammal.

### BACKGROUND OF THE INVENTION

Although recombinant viral vectors have shown great promise in overcoming a principal barrier to gene delivery, i.e., delivery of an exogenous gene inside a targeted cell, such vectors face major obstacles that limit the therapeutic application of gene-based medicines. For one, they are limited to genetic constructions inserted into the viral vector genome and to specific cell types according to their cell binding specificity determined by the viral “tropism”. Importantly, they face other major obstacles that limit their therapeutic application for example, immunogenicity of the viral vector, which not only adversely affects vector effectiveness but also causes significant toxicity problems. To this end, particles produced using a natural viral packaging cell often cause a patient's immune defense to mount a response to the administered viral vector particle. This “natural packaging” produces particles virtually identical to those of the virus from which the vector is derived. The produced viral capsid or envelop is based on the natural tropism of the virus which determines which tissues and cells are targets. Moreover, the proteinacious nature of the capsid and envelop is completely sensitive and susceptible to host immune defenses, which block the delivery of the recombinant genome. Toxicity resulting from the immune response also adds significantly to the problem.

The drawbacks of toxicity and immunogenicity particularly limit the use of viral vectors. This is particularly a problem where multiple administration of the vector is needed to achieve therapeutic effect. This problem also applies to use of viral vectors in vaccines, which require repeated, or booster, doses of a particular antigen. For example, the premature clearance of a vector from the body substantially eliminates the ability to use the vector to provide a boost by repeated administration of the vector containing the gene of interest. As a result, gene expression vaccine studies use boosts typically composed of an agent distinct from that used to prime the response. When a plasmid DNA is used to prime the response then the boost is provided by either the antigen protein itself or a viral vector capable of strong expression. Adenoviral vectors are often used since they have strong transduction capabilities for APCs (Rothel et al., Parasite Immunol. 1997 19, 221-7; Hammond et al., Vet. Microbiol. 2001 80, 101-19). Efforts to address this problem have resorted to administering a combination of plasmids, one conveying the genome of a virus with a different gene for its outer envelop protein taken from a different virus specific for a different species host (this change makes the virus unable to bind and infect human cells); and the other conveying the receptor needed by the new envelop protein (Matano et al., Vaccine 2000 18, 3310-8). These processes are cumbersome and expensive. Accordingly, there is a need for a gene delivery vehicle that is capable of effectively delivering an exogenous gene to a targeted cell, yet does not elicit a humoral or cellular immune response upon repeated interaction with the cellular environment.

Another drawback to administering live, attenuated viruses is the considerable safety risk they pose. While efforts have been applied to control viral replication mechanisms, certain levels of replication are needed to meet desirable efficacy levels for preventive vaccines. Nonetheless, viral replication represents the potential for severe toxicity when the aim of viral vectors is to achieve therapeutic efficacy derived from activity of the expressed gene in target cells and tissues. In the case of therapeutic effects derived from killing target cells or tissues, engineered cytolytic viral replication selective for the target cells and tissues has been studied. Thus therapeutic utility of viral vectors spans the range of replication level from complete elimination to strongly tissue selective. Hence, one of the clear challenges in achieving the desired therapeutic effect of gene expression is adequate delivery potency that still permits repeated administration, whether that expression is a therapeutic protein or is viral replication or a combination thereof and whether the intended effect is preventative, as in a vaccine, or therapeutic treatment.

Non-viral delivery systems have been developed to overcome the safety problems associated with live vectors. Although such non-viral systems generally are permissive of repeated administration and often are able to incorporate a wide variety of nucleic acid compositions, they frequently are limited by low efficiency and a very short persistence. Most of the non-viral delivery development has been with cationic lipid complexes and more recently cationic polymer complexes where the negatively charged plasmid DNA is bound and condensed with cationic molecules, usually studied with an excess of the cationic component. Many other chemical formulations have been studied including neutral polymers and simple aqueous solutions. The results obtained in these studies have revealed that effectiveness of gene delivery and expression by any one non-viral vector depends on the tissue and cells and route of administration. For example, injection of cationic lipid-plasmid complexes into the tail vein of mice results in widely varying gene expression in different organs but in all cases far greater than aqueous plasmid; lung expression levels are by far the greatest. On the other hand, cationic lipid complexes have been found to diminish gene expression, compared with aqueous plasmids, in muscle following intramuscular administration. Physical means to force plasmid DNA into cells in certain tissues also has shown promise. The use of gold particles with plasmid DNA on the surface has been used to bombard a tissue with DNA. Similarly, hydrodynamic pressure has been used to deliver plasmids into organs through the vascular bed. Also, once plasmid DNA has been delivered into muscle or skin by local administration, electroporation based on applied electric fields has been used to enhance delivery and expression.

For treatment of arthritis diseases of the joints, non-viral vectors have been studied using direct injection into the joint, where there is frequently a need to diminish inflammation. Unfortunately, the viral vectors and non-viral cationic complexes employed have exhibited a strong tendence to increase inflammation, thus severely reducing their effectiveness. The low level of expression obtained by aqueous plasmid, which reduces the level of exacerbated inflammation, has not satisfactorily addressed this major clinical need.

Another problem of non-viral vectors has been a dependence on plasmid DNA. The bacterial production of plasmid DNA poses several problems including use of antibiotic selection, bacterial origin of replication, residual bacterial proteins and lipid contaminants, and in particular a lack of methylation that occurs from mammalian cells. For therapeutic strategies dependent upon attenuated or controlled viral replication, plasmid DNA has been inadequate since it lacks replication capabilities for mammalian cells. Yet another limitation of plasmid DNA has been difficulty in expressing adequate levels of an RNA so as to achieve an antisense inhibition of an mRNA. Synthetic oligonucleotides have been developed that, in cell culture, exhibit inhibition of a specific gene according to its sequence. However, improved delivery of these nucleic acid agents is required in order to achieve an effective therapeutic effect. As a consequence, of these and other issues, there is a need to identify alternative nucleic acid payloads for non-viral vectors.

There is, accordingly, a need for improved nucleic acid delivery systems that: (i) are less toxic than conventionally used viral vectors, (ii) can be repeatedly administered, (iii) can be delivered to target cells and tissues without dependence on viral particle cell specificity, (iv) can be designed to provide required levels of viral replication, (v) can give strong expression in arthritic joints while minimizing any increase in inflammation, (vi) can deliver synthetic oligonucleotides in an effect amount to target cells and tissues, and (vii) provide for therapeutically effective levels of altered expression and prolonged persistence in vivo during subsequent readministration.

### SUMMARY OF THE INVENTION

Accordingly, it is an object of the present invention to provide methods of using gene delivery vehicles that are suitable for repeated in vivo administration.

It is another object of the invention to deliver a nucleic acid to a subject that leads to a therapeutic effect.

It is still another object of the invention to provide methods of administering a therapeutic agent to a subject in need thereof on a repeated basis.

It is a further object of the invention to provide enhancement of nucleic acid delivery using physical methods, such as electroporation.

These and other objects will become apparent to a skilled worker by reference to the specification and conventional teachings in the art.

In one aspect, the invention provides a method of obtaining a physiological response in a subject, by administering to the subject a dosage of a therapeutic nucleic acid molecule wherein the administered nucleic acid is an viral genome or comprises a viral genome sequence. In another aspect, the nucleic acid molecule may be administered in conjunction with electroporation. In another aspect, the administered nucleic acid that encodes the viral genomic sequence is capable of controlled levels of replication in vivo.

In another aspect, the invention provides a method of obtaining a physiological response in a subject, by administering to the subject a dosage of a therapeutic oligonucleic acid (antisense, ribozyme, siRNA, dsRNA) molecule wherein the administered nucleic acid inhibits the generation of a biological agent. In another aspect, the nucleic acid molecule may be administered in conjunction with electroporation.

The invention also provides a method of reducing inflammation in a subject suffering from a disorder characterized by inflammation, including the steps of: administering to the subject at, or proximal to, the site of the inflammation a therapeutically effective amount of a nucleic acid molecule that alters expression or activity of a polypeptide where the altered expression results in a desired therapeutic effect, wherein the administered nucleic acid is comprised within (i) a nucleic acid encoding a viral genomic sequence, (ii) a synthetic nucleic acid analog or conjugate, (iii) a DNA molecule, or (iv) an RNA molecule, and wherein the altered expression or activity of the nucleic acid alleviates the arthritic condition. The nucleic acid molecule may be administered in conjunction with electroporation. The inflammatory disorder may be selected from the group consisting of arthritis, gout and a localized bowel inflammatory disorder.

In another aspect, the invention provides a method of treating or alleviating the symptoms of a disease in a mammal, comprising administering a therapeutically effective amount of a nucleic acid composition to a tissue of the mammal, where the nucleic acid is comprised within a nucleic acid encoding a viral genomic sequence. The viral genomic sequence may be capable of repeated self-replication in vivo. The nucleic acid also may be comprised within a synthetic vector, and/or may be applied substantially contemporaneously with pulsed electric field to said tissue. The nucleic acid composition may reduce or increases the expression of a protein or polypeptide in the mammal. For example, the nucleic acid composition may decrease the expression of an oncogene, a protein kinase or a transcription factor, or may increase the expression of a tumor suppressor protein, an immunostimulatory cytokine or an oncolytic protein. The immunostimulatory cytokine may be, for example, GM-CSF, IL-1, IL-12, IL-1 5, an interferon, B-40, B-7, or tumor necrosis factor.

In another aspect the invention provides a method of treating or alleviating the symptoms of a disease in a mammal, comprising administering a therapeutically effective amount of a nucleic acid composition to a tissue of the mammal, where the nucleic acid is a single or double stranded oligonucleotide and wherein the nucleic acid is either (i) comprised within a synthetic vector, or (ii) applied substantially contemporaneously with a pulsed electric field to the tissue.. The method according to claim 9, wherein said nucleic acid composition reduces the expression of a protein or polypeptide in said mammal. The nucleic acid composition may be, for example, an antisense oligonucleotide, RNAi, or a non-naturally occurring oligonucleotide. The nucleic acid may reduce the expression of, for example, an oncogene, a protein kinase or a transcription factor. The protein or polypeptide may be, for example, BCL2, VEGF R2, NF kappa B, RAF kinase, PKC delta, HER2, or bFGF.

In still another aspect the method comprises a method of treating or alleviating the symptoms of a disease in a mammal, comprising applying a therapeutically effective amount of an anti-inflammatory composition into a joint of the mammal and substantially contemporaneously applying a pulsed electric field to the joint. The anti-inflammatory composition may comprise, for example, a nucleic acid, a small molecule drug, a peptide, or a protein. When the anti-inflammatory composition is a nucleic acid, it may be, for example, a single or double stranded DNA, RNA, a viral genome lacking a capsid protein, a synthetic non naturally occurring nucleic acid, or a single or double stranded oligonucleotide. The nucleic acid may be, for example, a DNA, RNA, or viral genome encoding at least one anti-inflammatory protein. The anti-inflammtory composition may be a single or double stranded oligonucleotide that decreases expression of a pro-inflammatory cytokine in the joint. The oligonucleotide may be non-naturally occurring oligonucleotide, or may be an RNAi.

### BRIEF DESCRIPTION OF THE DRAWINGS

FIG. 1 depicts fluorescent microscopy images showing cellular uptake of Rh-oligonucleotides complexed with different PEI reagents in HELA cells at charge ratio 6: PEI, PEI conjugated with PEG, and PEI-PEG with a peptide ligand (RGD) on the distal end of the PEG.

FIG. 2 provides expression measurements of pCI-LUC complexed with different PEI reagents: PEI, PEI conjugated with PEG, and PEI-PEG with a peptide ligand (RGD) on the distal end of the PEG.

FIG. 3 provides expression measurements of pCI-LUC when delivered by a combination of local administration and applied electric field and alone or in combination with inhibitor oligonucleotides into human xenograft tumors implanted subcutaneously.

### DETAILED DESCRIPTION OF THE INVENTION

Methods are provided for the efficient and sustained delivery of therapeutic compositions, for example, nucleic acids into joints. There methods are useful for treatment of diseases, such as osteoarthritis and rheumatoid arthritis, that afflict joint tissue. The compositions delivered using these methods preferably have anti-inflammatory activity, for example, proteins or polypeptides having anti-inflammatory activity or nucleic acids that encode such proteins. The compositions are delivered to the joint tissue in conjunction with electroporation, which significantly enhances efficiency of the delivery.

Methods also are provided for delivery of viral genomic constructs and oligonucleotides into any tissue or cell of a mammal. Delivery of the viral genomic constructs and oligonucleotides is enhanced by the use of synthetic vectors and/or electroporation. These methods are suitable for efficient delivery of viral genomic constructs and oligonucleotides into tissues including, but not limited to, muscle, tumor, and skin. The oligonucleotides suitable for use in these methods include, but are not limited to, single and double stranded RNA, DNA, mixtures of RNA and DNA, and non-naturally occurring molecules such as peptide nucleic acids, as discussed in more detail below. These methods may be used to treat a wide range of diseases and disorders in mammals, and particularly in humans.

It has been found that certain nucleic acid delivery vehicles can be used to administer to a subject an effective amount of a therapeutic nucleic acid molecule and repeatedly over the extended period of time required for benefit. This finding is significant, given the adverse cellular and humoral immune response against the administered viral vector that typically accompanies a regimen of repeated attempts of gene delivery. As a result, the nucleic acid delivery vehicles of the invention can be useful in a number of therapeutic applications, including, for example: therapeutic vaccines, treatment of inflammatory disorders and many types of malignancies, as well as any other regimen involving repeated administration or expression of a therapeutic nucleic acid molecule or polypeptide.

Preferably, a nucleic acid delivery vehicle for use in the present invention exhibits two properties. First, it should have the ability to deliver a therapeutic amount of one or more nucleic acid molecules in vivo, e.g., to a mammalian system. In this regard, delivery of the vehicle can be aided by techniques such as, e.g., electroporation. Second, it should be able to deliver the therapeutic nucleic acid molecule without stimulating an unwanted immune response that causes substantial and/or premature clearance of the nucleic acid delivery vehicle from the in vivo system.

Upon delivery of nucleic acid into a targeted cell or tissue, a vehicle used according to one embodiment of the present invention is capable of enabling attenuated or controlled replication, thereby providing a therapeutic amount of a nucleic acid molecule throughout cells of a tissue and/or for an extended period of time. Additionally, in another embodiment the vehicle is capable of delivering sequence specific oligonucleotide inhibitors.

Non-Immunostimulatory Characteristics

The present invention provides nucleic acid delivery methods that do not stimulate an immune response to the same degree typically associated with conventionally available vectors. For example, when conventional viral vectors or cationic complexes of plasmids are injected into joints, an increase in indicators of inflammation, such as neutrophil levels, is observed. The administration of aqueous nucleic acid does not induce this imunostimulation but lacks the ability to provide adequate nucleic acid activity. The combination of aqueous nucleic acid with applied electric fields as achieved by electroporation achieves delivery of the nucleic acid while minimizing induced inflammation. Similarly, synthetic vectors that have a component blocking non-specific interactions with cells and tissues but having specific, i.e. selective, activity for target cells and tissues overcomes unwanted immunostimulation. Suitable delivery vehicles comprise (i) a therapeutic nucleic acid molecule, together with (ii) a synthetic reagent, and/or (iii) transiently applied electrical fields. Each of these delivery vehicles is less immunogenic than viral vectors since they lack the natural proteinacious capsid or envelop of viral vectors. They are also less immunogenic than cationic complexes of plasmids since they lack the non-specific interactions that trigger immune response in concert with the desired activities.

Nucleic Acids Encoding a Viral Genomic Sequence

In one aspect, the invention contemplates using isolated and, in some instances, purified nucleic acid molecules that can encode all or parts of a viral genomic sequence (also described herein as a “viral genome”) or sequences matching viral genomic sequences. Viral genome-encoding nucleic acids for use in the present invention are less antigenic than conventional viral vectors, since the former do not provide antigenic capsid proteins. This viral genome may be isolated from viral vectors and may be capable of replicating in a controlled or tissue specific manner.

The replication may be achieved by tissue specific or selective promoters driving expression of viral proteins and replication once the nucleic acid is delivered to the target cells and tissue. Since viral genomes (or a portion thereof) can be replicated in mammalian cells, nucleic acid molecules encoding the genome can be engineered to deliver and express nucleic acids engineered to alter the levels or activity of therapeutic polypeptides. Replication of the viral genome will result in the replication of the therapeutic gene thereby amplifying the effect of the therapeutic agent. Such viral genome-encoding nucleic acid molecules include, for instance, adenoviral genomic DNA-protein conjugates, alphaviral genomic RNA molecules, retro or lentiviral genomic RNA, and adeno-associated DNA.

Incorporation of oncolytic adenoviral genomic DNA sequences into a nucleic acid can be used to combine non-viral delivery systems with in vivo oncolytic replication. For example, isolated oncolytic adenoviral DNA can be delivered into tumors and its delivery further facilitated by application of electric fields to the tumor by use of electroporation. In another example, tumor selective promoter driven oncolytic adenoviral replication can be incorporated into a plasmid and then delivered into tumors using local administration combined with electroporation.

Clearly, expression of a therapeutic nucleic acid by a promoter can be combined in cis or in trans with nucleic acids containing viral genomic sequences. In this embodiment, activity of the therapeutic nucleic acid can be amplified by replication or can be combined with a separate therapeutic activity contained within the viral genomic sequences, for example, oncolytic replication of the viral genomic sequence.

Adenoviral genomic DNA is a suitable candidate for the viral genome sequence, since the naturally conjugated Terminal Protein-DNA form of this DNA molecule confers nuclear targeting, episomal stability, and other beneficial properties, which are desirable for use in the present invention. According to the invention, a viral vector genome (e.g., adenovirus) can be utilized as a nucleic acid since the design of the viral vector deletes early gene sequences which are required for initiation of replication making the vector attenuated in normal mammalian cells that lack complementary proteins for the deleted sequences.

Alternatively, a viral vector genome may contain sequences allowing replication only in certain tissue(s). In this case, the deleted element controlling initiation of viral replication is replaced by a mammalian form of the element but that is restricted to cells of the target tissue. Thus, viral replication is restricted to those cells of the target tissue that contain the complementary element.

One example of this latter embodiment is a gene delivery vehicle comprising a granulocyte macrophage colony stimulating factor (GM-CSF) expression cassette, which is incorporated into a viral vector genome (e.g., adenoviral) defective in the adenoviral E1B early gene. This viral gene inactivates RB in mammalian cells and allows viral replication to initiate. The lack of this early gene renders the resulting genome unable to replicate in normal cells, but able to replicate in tumor cells with a mutant tumor specific form of RB incapable of blocking initiation of viral replication allowing replication even without E1B in these tumor cells. Another form of adenoviral vector genome supporting tissue-selective replication utilizes a tissue-selective promoter for the viral E1 gene activity required for initiation of viral replication. Suitable promoters are selectively active in target cells and sufficiently active to initiate viral replication.

An alphaviral vector genomic RNA molecule also can be utilized in accordance with the invention. Alphaviral RNA expression occurs in the cytoplasm, providing for (i) conversion of the genomic RNA into RNA, i.e., mRNA, from which peptides and proteins can be synthesized, and (ii) production of peptides and proteins. The conversion of genomic RNA into mRNA preferably results in a large pool of mRNA, which, in turn, allows for amplification of peptide and protein expression. Examples of suitable alphavirus genomes include Sindbis and Semliki forest virus. See also Wahlfors et al. “Evaluation of recombinant alphaviruses as vectors in gene therapy.” Gene Ther 2000:7:472-480.

In addition, generation of viral particles from the encoded viral genome may permit transfer of an administered therapeutic nucleic acid to other cells and tissues; in particular, neighboring cells and tissues. Once the viral particles are generated in a cell, these particles can come out of these cells and bind to and enter neighboring cells if the viral particles produced have the appropriate tropism for the cells. When the particles have adequate activity to spread to neighboring cells, their production thereby causes the propagation of the nucleic acid delivery effect. Thus, the invention provides for delivery of a nucleic acid encoding a viral genome (or viral particles thereof), which can provide for secondary production of protein and in some instances nucleic acids, which may provide for yet further nucleic acid or protein production. This “replicative” aspect of the viral nucleic acid can provide an extended or ongoing supply of one or more therapeutic acids without having to re-administer a therapeutic supply of the nucleic acid comprised within a nucleic acid delivery vehicle. However, if re-administration is necessary, administration of the genomic DNA repeatedly will be possible since it will not be affected by the antibody generated against the viral capsid proteins in the subject.

Other Forms of Nucleic Acids

The methods of the invention also can utilize other forms of nucleic acids such as synthetic or non-naturally occurring forms of nucleic acid, such as phosphorothioate antisense oligonucleotides, aptamers, siRNA, or double strand RNAi, and chemical derivatives of the nucleic acids that are well known in the art. Examples include conjugates of nucleic acids with peptides and proteins, chemical derivatives of the nucleic acid ribose-phosphate backbone such as phosphorothioates and 2′ methyoxy-ethoxy ribose, morpholino, and peptide-nucleic acids, wherein the bases are appended to a peptide backbone. In other instances, the invention provides for complexes of nucleic acids. Examples of complexes include antisense and triplex oligonucleotides bound to matching sequences. In some cases such molecules can be incorporated in viral genomes where the oligonucleotide is expressed by transcription but in this case only natural forms are expressed.

Synthetic Vectors

The invention also contemplates using one or more synthetic vectors as a nucleic acid delivery vehicle. Synthetic vectors for use in the present invention have been disclosed by Woodle et al.(WO 01/49324, filed Dec. 28, 2000). This application is hereby incorporated by reference in its entirety, including the drawings.

As used herein, a “synthetic vector” means a multi-functional synthetic vector which, at a minimum, contains a nucleic acid binding domain and a ligand binding (e.g. tissue targeting) domain, and is complexed with a nucleic acid sequence. A synthetic vector also may contain other domains such as, for example, a hydrophilic polymer domain, endosome disruption or dissociation domain, nuclear targeting domain, and nucleic acid condensing domain. A synthetic vector for use in the present invention preferably provides reduced non-specific interactions, yet effectively can engage in ligand-mediated (i.e. specific) cellular binding. In addition, a synthetic vector for use in the present invention is able to be complexed to one or more therapeutic nucleic acids, which then can be administered to a subject.

The nucleic acid binding domain, or “complex forming reagent,” can associate with a core nucleic acid complex in a manner that allows assembly of the nucleic acid core complex. The complex forming reagent can be, e.g., a lipid, a synthetic polymer, a natural polymer, a semi-synthetic polymer, a mixture of lipids, a mixture of polymers, a lipid and polymer combination, or a spermine analogue complex, though the skilled artisan will recognize that other reagents may be used.. A suitable polymer may contain histidine or an imidazole functional group. WO 01/49324 at, e.g., pages 20-34 disclose suitable DNA binding domains for use in the present invention.

The complex forming reagent preferably has an affinity sufficient to enable formation of the complex under the conditions present for the preparation and sufficient to maintain the complex during storage and under conditions present following administration but which is insufficient to maintain the complex under conditions in the cytoplasm or nucleus of the target cell. Common examples of complex-forming reagents include cationic lipids and polymers, which permit spontaneous complexation with the core nucleic acid moiety under suitable mixing conditions, although neutral and negatively charged lipids and polymers may be used. Other examples include lipids and polymers in combination where some are cationic in nature and others in the combination are neutral or anionic in nature such that together a complex with a desired stability balance is attained. In yet other examples, lipid and polymers may be used that have non-electrostatic interactions but that still enable complex formation with a desired stability balance. For example, the desired stability balance may be achieved through interactions with nucleic acid bases and back bone moieties like those of triplex oligonucletide or “peptide nucleic acid” binding. In yet further examples conjugated lipids and polymers alone and in combinations may be used.

Suitable cationic compounds also include spermine analogues. The core complex formed with spermine analogues preferably comprises membrane disruption agents. In another embodiment, the core complex formed with spermine analogues comprises anionic agents to convey a negative surface charge to the core complex.

Suitable polymers for use in the invention include polyethyleneimine (PEI), and advantageously PEI that is linear, polylysine, polyamidoamine (PAMAM dendrimer polymers, U.S. Pat. No. 5,661,025), linear polyamidoamine (Hill et al., Linear poly(amidoamine)s: physicochemical interactions with DNA and Biological Properties, in Vector Targeting Strategies for Therapeutic Gene Delivery (Abstracts form Cold Spring Harbor Laboratory 1999 meeting), 1999, p 27), protamine sulfate, polybrine, chitosan (Leong et al. J Controlled Release 1998 Apr; 53(1-3):183-93), polymethacrylate, polyamines (U.S. Pat. No. 5,880,161) and spermine analogues (U.S. Pat. No. 5,783,178), polymethylacrylate and its derivatives such as poly[2-(diethylamino)ethyl methacrylate] (PDEAMA) (Asayama et al., Proc. Int. Symp. Control. Rel. Bioact. Mater. 26, #6236 (1999) and Cherng et al. Eur J Pharm Biopharm 47(3):215-24 (1999)) and poly(2-(dimethylamino) ethyl methacrylate) (PDMAEMA) (van de Wetering et al., J Controlled Release 53:145-53(1998)), poly(organo)phosphazenes (U.S. Pat. No. 5,914,231), which are hereby incorporated by reference in their entirety. Other polymers that may be used in the complex include polylysine, (poly(L), poly(D), and poly(D/L)), synthetic peptides containing amphipathic aminoacid sequences such as the “GALA” and “KALA” peptides (Wyman T B, Nicol F, Zelphati O, Scaria P V, Plank C, Szoka F C Jr, Biochemistry 1997, 36:3008-3017; Subbarao N K, Parente R A, Szoka F C Jr, Nadasdi L, Pongracz K, Biochemistry 1987 26:2964-2972) and forms containing non-natural aminoacids including D aminoacids and chemical analogues such as peptoids, imidazole-containing polymers, and fully synthetic polymers that bind and condense nucleic acid. Assays for polymers that exhibit such properties include measurements of plasmid DNA condensation into small particles using physical measurements such as DLS (dynamic light scattering) and electron microscopy.

The core complex advantageously will be self-assembling when mixing of the components occurs under appropriate conditions. Suitable conditions for preparing the core complex generally permit the charged component that is present in charge molar excess at the end of the mixing to be in excess throughout the mixing. For example, if the final preparation is a net negative charge excess then the cationic agent is mixed into the anionic agent so that the complexes formed never have a net excess of cationic agent. Another suitable condition for preparing the core complex utilizes a continuous mixing process including mixing of the core components in a static mixer. A static mixer produces turbulent flow and preferably low shear force mixing in two or more fluid streams flowing into and through a stationary device resulting in a mixed fluid that exits the device. For core complexes low shear force mixing is expecially important when the nucleic acid is fragile to shear. Specifically, aqueous solutions of nucleic acid and core complex-forming moieties (such as a cationic lipid) are fed together into a static mixer (available from, for example, American Scientific Instruments, Richmond, Calif.), where the streams are split into inner and outer helical streams that intersect at several different points causing turbulence and thereby promoting mixing. The use of commercially available static mixers ensures that the results obtained are operator-independent, and are scalable, reproducible, and controllable. The core complex particles so produced are homogeneous, stable, and can be sterile filtered. When the core complex is intended to contain a nuclear targeting moiety and/or a fusogenic moiety, these components may be added directly into the streams entering the static mixer so that they are automatically incorporated into the core complex as it is formed.

In one embodiment of the invention, the use of core complexes which are negative or neutral in surface charge is preferred. In this embodiment, the outer shell conveys target tissue and cell binding and uptake properties in contrast to the cationic complex-anionic cell electrostatic binding mechanism that is thought to provide binding and uptake by positively-charged core complexes. By allowing use of neutral or negative surface charge core complexes, numerous benefits can be realized. The reduction or elimination of electrostatic interactions with positive surface charge vector colloids can reduce or eliminate non-specific interactions leading to phagocytic clearance, to toxicity in non-target tissues and organs, and to cell toxicity in target tissues and organs.

In one embodiment, the therapeutic nucleic acid is comprised within a nucleic acid sequence encoding a viral genomic sequence; the entire nucleic acid sequence (i.e. comprising both the viral genomic sequence and therapeutic nucleic acid sequence) is complexed to the DNA binding domain of the synthetic vector. For example, isolated adenoviral vector genome can be constructed with an insertion of an expression cassette for a therapeutic transgene in an E3 deleted region of the adenoviral vector. The isolated genome is then delivered using a synthetic vector and/or electroporation. Alternatively, the entire nucleic acid sequence can be cloned into a plasmid to generate multiple copies, then the plasmid can be complexed to the DNA binding domain of the synthetic vector. In either embodiment, the nucleic acid encoding a viral genome can be replicative once administered to a subject, thereby providing an ongoing supply of a therapeutic nucleic acid molecule. The replicative nature is controlled by use of tissue selective promoters for initiation of replication or by tumor cells with mutations that allow replication even when viral elements for initiation of replication are deleted.

The vectors of the present invention may be used to deliver essentially any nucleic acid that is of therapeutic or diagnostic value. The nucleic acid may be a DNA, an RNA, a nucleic acid homolog, such as a triplex forming oligonucleotide or peptide nucleic acid (PNA), or may be combinations of these. Suitable nucleic acids may include, but are not limited to, a recombinant plasmid, a replication-deficient plasmid, a mini-plasmid lacking bacterial sequences, a recombinant viral genome, a linear nucleic acid fragment encoding a therapeutic peptide or protein, a hybrid DNA/RNA double strand, double stranded RNA, an antisense DNA or chemical analogue, an antisense RNA or chemical analogue, a linear polynucleotide that is transcribed as an antisense RNA or a ribozyme, a ribozyme, and a viral genome.

A synthetic vector for use in the invention can be used to target specific tissues. In the absence of a steric coat, the cationic surface charge of a synthetic vector can act to target a cell. The ability to bind a target cell can be lost when a steric polymer coat is added to the synthetic vector as a hydrophilic polymer domain, such as a hydrophilic polymer domain disclosed herein. Targeting activity of the synthetic vector can be restored by employing a ligand domain, which can effectuate ligand-mediated binding and cellular uptake of the synthetic vector. In one aspect, a ligand may be conjugated to the distal end of the steric polymer in order to mediate binding with one or more cell surface receptors.

The invention contemplates using any conventionally available ligand domain as part of a synthetic vector, provided that it does not inhibit delivery and expression of the therapeutic nucleic acid. Example 2, for instance, utilizes cyclic RGD peptide as a ligand domain, which can be conjugated onto a steric polymer conjugate, using condensing agents. Synthetic vector constructs containing cyclic RGD have demonstrated strong binding and delivery to cancer cells. Other tri-block conjugates are envisioned for use in the invention, however. Other examples include transferrin, folate, YIGSR, sialy Lewisₓ, and cell-binding peptides. Suitable cell-binding peptides with desired binding abilities can be identified by methods well known in the art, for example, by phage display.

The synthetic vectors for use in the invention also account for drawbacks associated with other nucleic acid delivery vehicles, such as non-specific interactions, which often result from an electrostatic charge differential between a vector and its environment. A synthetic vector, e.g., a condensed cationic reagent-DNA complex, can be made net positive, neutral, or negative depending on the ratio of the components in the complex. While electrostatic interactions between a negatively charged cell membrane and a positively charged particle can increase cellular uptake, all cells possess a negative membrane charge. Accordingly, non-specific interactions can persist in a complex containing a net positive charge.

A hydrophilic polymer domain of a synthetic vector preferably is able to minimize undesirable non-specific interactions by controlling the surface properties of the synthetic vector. The hydrophilic polymer may be selected from the group consisting of poly(ethyleneglycol) (“PEG”), polyoxazoline, HPMA, polyacetal and other conventionally known hydrophilic polymers. Such polymers can shield the net positive charge of complexed nucleic acid, and thereby reduce unwanted non-specific interactions.

The outer steric layer preferably comprises a hydrophilic, biodegradable polymer. If the hydrophilic polymer is not biodegradable then a relatively low molecular weight (<30 kDaltons) polymer is used. The polymer may also exhibit solubility in both polar and non-polar solvents. Suitable polymers include PEG (of various molecular weights), polyvinylpyrrolidone (PVP), and polyvinylalcohol, polyvinylmethylether, polyhydroxypropyl methacrylate, polyhydroxypropylmethacrylamide, polyhydroxyethyl acrylate, polymethacrylamide, polydimethylacrylamide, polylactic acid, polyglycolic acid, polymethyloxazoline, polyethyloxazoline, polyhydroxyethyloxazoline, polyhydroxypropyloxazoline, or polyaspartamide which are well known in the art (U.S. Pat. No.5,631,018).

Other suitable polymers include those that will form a steric barrier on colloidal particulates of at least 5 nm “thickness” or greater as determined by reduction in zeta potential (Woodle et al., Biophys. J. 61:902 (1992)) or other such assays. Further suitable polymers include those that contain branches. In one embodiment, the hydroxyl functions of a glucose moiety are used to conjugate multiple steric polymers, one of which is anchored to the core complex. In another embodiment, the amine functions of a lysine are used to conjugate two steric polymers and the carboxyl function is used with a steric polymer linker to conjugate onto the core complex.

A hydrophilic, steric coat can be introduced onto the surface of a synthetic vector by covalently conjugating the polymer to the condensing agent before complexing with therapeutic nucleic acid. This method is preferred over conjugating a hydrophilic, steric polymer to a pre-formed DNA-synthetic vector complex, since chemical reactions carried out after DNA complexing can damage the DNA. Moreover, as the steric barrier is formed, subsequent conjugation reactions are inhibited, which can limit the amount of polymer that can be conjugated to the complex surface.

In one example, a hydrophilic polymer is conjugated at random to one or more sites on the nucleic acid binding domain, using either a stable covalent linkage or a linkage that can be cleaved. Such linkages include disulfide bonds, esters, hydrazones, and vinyl ethers . The grafting density can be varied between 2% and 25% of monomer units (for polyetherimide (“PEI”), this is amines). Samples having a single molecular weight of the steric polymer can be used. An alternative steric polymer is polyacetal derived by oxidation and subsequent reduction of dextran. The polymer is linear, possessing one or two alcohol moieties in place of each hexose ring. Polyacetal has been shown to function as a steric polymer for drug delivery and when conjugated to lipids and polycations.

A steric polymer layer that can block non-specific binding can increase the serum half-life of a synthetic vector, since (i) minimal non-specific interactions render the particles relatively inert, and (ii) the relatively large size allows the synthetic vector to remain in the blood for prolonged periods. Successful construction and use of a steric polymer layer can be observed from blood pharmacokinetics of the complexes following an intravenous administration (e.g., PEG, PEI and DOTAP:Cholesterol complexes). PEG, a leading steric polymer candidate for liposomes, has been shown to provide protection for nucleic acid complexes. As illustrated in Example 2, a steric polymer layer (PEG) added to the surface of a synthetic vector complex surface rendered the complex significantly inactive, as expected.

Enhanced Delivery of a Therapeutic Nucleic Acid

Enhanced delivery of a nucleic acid delivery vehicle also can be effectuated by altering one or more delivery parameters. For instance, enhanced delivery can involve application of an electric field, alteration in hydration or hydrostatic pressure, inclusion of excipients, and/or variation in pH or buffering of pH in the cellular environment.

Application of transient electrical fields can be varied in several parameters including pulse duration, voltage, number of pulses, timing between pulses, variation in properties of each pulse in a series of pulses, use of penetrating or non-penetrating electrodes, patterns of electrodes, patterns of voltage pulses applied to specific electrodes, and surface properties of electrodes such as those affecting current flow.

Hydration levels can be varied in several parameters including salts and pH buffering, volume injected, route of administration, needle, rate of injection, and excipients such as hydrophilic polymers, and biological response modifiers such as bradykinin and nuclease inhibitors. Excipients that can be used include those that form a controlled release depot such as microspheres and hydrogels, those improve stability (e.g., physical and/or biological state) of a therapeutic nucleic acid such as nuclease inhibitors and non-ionic polymers such as polyvinylpyrrolidone (PVP), and those that facilitate trafficking through the tissue and binding target cell types such as ligand bearing polymers with imidazole moieties having weak pH sensitive binding to the nucleic acid.

In a preferred embodiment, enhanced nucleic acid delivery occurs by administering a nucleic acid delivery vehicle to a cellular environment in conjunction with application of an electric field often called electroporation. As used herein, “electroporation” means a transiently applied electric field or series of transiently applied electric fields applied across target cells and tissues exposed to the therapeutic nucleic acid either before or shortly after application of the electric field. The enhanced delivery by electric fields can be a result of penetrating electrodes, non-penetrating electrodes or a combination thereof. The electrodes can be arranged as a pair or as many electrodes. The polarity of the voltage can be reversed or varied to increase exposure of as many cells and tissues as possible to the transient applied electric field. In addition, enhanced delivery can result from low voltage pulses, high voltage pulses or a combination thereof and from long pulses, short pulses, or a combination thereof. The enhanced delivery also may be a result of low current flow, high current flow, or a combination of both through the region. A nucleic acid delivery vehicle administered in conjunction with electroporation can be administered to the general vicinity of the cells or the vehicles may be specifically targeted to the cells and tissues, which are exposed to electric fields. Endoscopic devices can be utilized to provide electrodes for applying an electric field.

According to the invention, electroporation can be utilized to deliver nucleic acids, including conventional plasmid DNA, to compartments such as synovial tissue and cells in joints, lung tissue, breast tissue, colon tissue, skin tissue, muscle tissue, bladder tissue, prostate tissue, the peritoneal cavity, tumors growing in tissues, blood vessels, the spinal column, isolated organs, and others. Conventional uses of electroporation are described by Heller, et al. (2000), Gene Therapy, 7:826-829; Heller, et al. (2001) DNA Cell Biol., 20(1):21; and Heller, et al. (2000) Melanoma Res., 10(6):577-83, each of which hereby is incorporated by reference.

A preferred embodiment of electroporation enhancement of nucleic acid delivery for enhanced tumor delivery utilizes pairs of non-penetrating electrodes positioned on either side of the tumor mass. Injection of the nucleic acid therapeutic agent into the tumor is followed by application of a series of long low voltage pulses with reversing polarity followed by a series of short high voltage pulses with reversing polarity. In yet another preferred embodiment of electroporation enhancement of nucleic acid delivery for enhanced tumor delivery utilizes roughly circular patterns of penetrating electrodes with a count of even multiples of four that are placed into the tumor mass either before or after administration of the nucleic acid. A series of long low pulses is applied followed by a series of high short pulses where the voltage is applied across at least two pairs of roughly parallel electrodes in the same polarity, followed by at least one pulse with reversed polarity and then followed by application of the voltage across at least two pairs of electrodes with an angle at least about 45 degrees from the previous applied voltage. The process is repeated until the desired level of nucleic acid uptake or biological activity is achieved. In yet another embodiment of the previous method the voltage is applied in opposite polarity between the two pair of electrodes operative at the same moment.

The foregoing enhanced delivery regimens can be utilized with any nucleic acid delivery vehicle contemplated herein, e.g., a synthetic vector or a viral genomic nucleic acid molecules encoding a viral genome, viral particles or both, or DNA, RNA, or non-naturally occurring nucleic acids and their conjugates.

Therapeutic Methods

The present invention provides methods of administering one or more therapeutic nucleic acid molecules to a subject, using a nucleic acid delivery vehicle with or without enhancement of delivery, to bring about a therapeutic benefit to the subject. As used herein, a “therapeutic nucleic acid molecule” or “therapeutic nucleic acid” is any nucleic acid (e.g., DNA, RNA, non-naturally occurring nucleic acids and their analogues such as peptide nucleic acids, and their chemical conjugates) that, as a nucleic acid or as an expressed nucleic acid or polypeptide, confers a therapeutic benefit to a subject. In the present invention, a therapeutic nucleic acid molecule is administered to a subject as part of, or via, a nucleic acid delivery vehicle. The subject preferably is mammalian such as a mouse, and more preferably is a human being.

Nucleic acid delivery vehicles for use in the present invention can be used to achieve a therapeutic response in a number of ways, including by increasing the levels of a polypeptide, by decreasing the levels of a polypeptide, by increasing or decreasing the levels of a therapeutic activity such as a kinase or transcription factor, or by stimulating or inhibiting an immune response, which may be protective or therapeutic. In this sense, the invention provides methods of enhancing or inhibiting a physiological response against an antigen in a subject.

The administration regimen can vary, depending on, for example, (i) the subject to whom the therapeutic nucleic acid molecule is administered, and (ii) the therapeutic need. For instance, a melanoma or head and neck cancer therapeutic may be treated by weekly administrations using skin penetrating electrodes for a period of weeks or months. Similarly, an ovarian, lung, or bladder cancer therapeutic may be treated by monthly administrations using an endoscope for a period of months. The regimen and route can be selected so as to achieve adequate expression or inhibition of the polypeptide or biological activity and repeat of the administration at a time when the initial therapeutic effect is weakening until the therapeutic effect is no longer desired or needed.

In the preceding administration steps, the administered nucleic acid molecule is comprised within or complexed to a nucleic acid delivery vehicle of the invention. Preferably, expression of the therapeutic nucleic acid molecule in the foregoing steps. elicits a therapeutic response including but not limited to increased or decreased levels of a polypeptide, increased or decreased levels of a biological activity, or modification of an immune response such as increased or decreased inflammation or a humoral and/or cellular response in the subject. In one embodiment, the therapeutic nucleic acid molecule may be administered in conjunction with a regimen of electroporation as described above.

In yet another embodiment, the invention provides for selective gene expression through use of tissue selective replication of viral nucleic acid. The invention provides for viral vector replication whereby viral vector particles are produced by the target cells and tissues. The viral vector particles so produced may or may not provide for tissue selective spread and amplification. In one embodiement, the invention provides for selective replication of a viral vector in tumor cells and tissues that provides for selective spread in the tumor cells and tissues and thereby amplifying the therapeutic effect on the tumor. For instance, expression of a therapeutic RNA inhibitory to tumor cells by a viral vector that spreads selectively in tumor cells and tissues can amplify the therapeutic effect of a treatment for cancer.

An administered therapeutic nucleic acid molecule also may induce an immune response. In one embodiment, the therapeutic nucleic acid encodes a cytokine, which may be expressed with or without an antigen. A cytokine acts to recruit an immune response, which can enhance an immune response to an expressed antigen. Accordingly, cytokine expression can be obtained whereby APCs and other immune response cells are recruited to the vicinity of tumor cells, in which case there is no requirement for co-expression of an antigen by the nucleic acid delivery vehicle. In another embodiment, one or more antigens and cytokines can be co-expressed.

Accordingly, the invention contemplates the use of immunostimulatory cytokines, as well as protein analogues exhibiting biological activity similar to an immunostimulatory cytokine.. Suitable cytokines for use in enhancing an immune response include GM-CSF, IL-1, IL-12, IL-15, interferons, B-40, B-7, tumor necrosis factor (TNF)and others. The invention also contemplates utilizing genes that down-regulate immunosuppressant cytokines.

The invention also provides for expression of “recruitment cytokines” at tumors. Expression of cytokines at tumors can recruit immune response cells and initiate a cellular immune response at the tumor site, thereby initiating immune recognition and killing of tumor cells both at the site of expression and at distal tumor sites. A preferred embodiment of the invention is comprised of (i) an adenoviral genomic nucleic acid, (ii) a nucleic acid exhibiting expression of GM-CSF under a tumor-preferential promoter, and (iii) a nucleic acid exhibiting tumor-conditional replication to form adenoviral vector particles exhibiting tumor-conditional replication. These nucleic acids are delivered using either a synthetic vector composition targeting delivery to tumor lesions, and/or via electroporation. Another preferred embodiment of the invention utilizes an adenoviral genomic nucleic acid encoding a cytokine (e.g., GM-CSF) under regulation of a tumor-conditional promoter. This feature would result in enhanced cytokine expression at the site of a tumor. In this embodiment, the adenoviral genomic nucleic acid preferably is administered in conjunction with electroporation to tumor lesions. For instance, a tumor selective replication competent adenoviral genome with a tumor selective promoter for E 1 A can have a mammalian expression cassette for IL-12 in a deleted region of E3. This viral genome is administered into tumor tissues followed by application of electric fields to the tumor tissues.

A nucleic acid delivery vehicle also may be used to treat a disorder characterized by inflammation. In one approach, one or more therapeutic nucleic acid molecules comprised within a nucleic acid delivery vehicle is administered to a subject suffering from a disorder characterized by inflammation, in order to suppress or retard an immune response. Treatable disorders include rheumatoid arthritis, psoriasis, gout and inflammatory bowel disorders.

Suitable therapeutic nucleic acids for use in treating inflammation include nucleic acids that encode an inflammation inhibitory cytokine. Examples for use in the present invention include IL-1RA, soluble TNF receptor, soluble Fas ligand, and the like.

The route and site of administration will vary, depending on the disorder and the location of inflammation. The nucleic acid delivery vehicle can be administered into a joint; administration thereto can be in conjunction with electroporation.

Nucleic acid delivery vehicles also can be used to treat cancer, cardiovascular diseases, viral and bacterial infections.

For therapeutic applications (cancer): injection of viral genome, plasmid, RNAi, antisense, or other nucleic acid therapeutics into tumor and in combination with electroporation of the tissue. Inhibitors of polypeptide expression such as antisense and RNAi can be used to reduce levels and biological activity giving a therapeutic effect such as inhibition of BCL2, VEGF R2, NF kappa beta, RAF kinase, PKC delta, HER2, bFGF, and others. The methods can also be used to express a tumor suppressor protein, such as p53, RB, DCC, and other tumor suppressors well known in the art. The methods of the present invention also include modalities wherein other therapeutic compositions are delivered to joint tissue using electroporation. In addition to the nucleic acid molecules described above, the electroporation methods can be used to directly administer agents such as peptides, small molecule drugs, proteins, and other therapeutic moieties well known in the art. Agents that have anti-inflammatory properties are particularly useful in this regard. Suitable anti-inflammatory agents are known in the art.

### EXAMPLES

The following examples are intended to be illustrative only and, accordingly, do not limit the scope of the invention thereto.

### Example 1:

### PEI-PEG Conjugates and Effect of PEGylation on the Size and Stability of PEI/DNA Complexes

PEI (25 kD) was obtained from Aldrich Chemical Company (Milwaukee, Wis.) and Methoxy poly (ethylene glycol)-nitrophenyl carbonate (MW 5000) from Shearwater Polymers (Birmingham Ala.). Concentration of PEI solutions was determined using a colorimetric TNBS assay for primary amine content. DNA concentration was determined spectrophotometrically using a molar extinction coefficient of 13,200 mol-1 cm-1 per base pair at 260 nm (10D=50 μg DNA). Particle size of DNA complexes was determined by light scattering with a Coulter N4 instrument. PEI-PEG conjugates were prepared by standard chemical methods. Briefly, 10 mg of PEI was dissolved in 100 mM NaHCO3 at pH 9 and 61 mg of methoxy-PEG5000-nitrophenyl carbonate (sufficient to modify 5% of PEI residues) added and allowed to react for 16 hours at 4° C. The reaction mixture was then dialyzed extensively against 250 mM NaCl followed by water using a dialysis bag with a 10,000 MW cut-off. Synthesis of PEI conjugate of PEG350 was carried out using a similar procedure as described for PEG5000 using nitrophenyl carbonates of PEG350, obtained from Fluka, Milwaukee, Wis. The extent of PEG conjugation was estimated using the weight of the complex and the concentration of primary amine.

Complexes of DNA/PEI-PEG containing various molar concentration of PEG were prepared by hand mixing of equal volumes of DNA and PEI/PEI-PEG mixtures, followed by vortexing for 30 to 60 seconds. PEG-conjugated PEI was dissolved in an aqueous solution to obtain a final concentration of 100 mM amine as determined by an ethidium bromide displacement assay. In this assay 1 mmol is defined as the amount of amine required to completely neutralize 1 mmol of DNA phosphate. From a 2.72 mg/ml stock solution of plasmid DNA (pCIIuc) 221 μl was combined with 110 μl of a concentrated aqueous solution of salts, buffers, detergents, etc. and 597 μl of water. 72 μl of the PEI solution was added to the mixture and vortexed thoroughly for 20 sec, to prepare complexes that have a 4:1 +/− ratio. The particle size and distribution of size for each preparation made was determined.

The effect of PEG on the cellular uptake of PEI/DNA complexes was evaluated by fluorescence microscopy. A 3′- Rhodamine labeled phosphorothioate oligonucleotide (5′-AAG GAA GGA AGG-3′-Rhodamine) obtained from Oligos Etc., Wilsonville, Oreg., was used as the fluorescent marker. The labeled oligonucleotide was complexed with PEI or PEI-PEG at 4:1 (+/−) charge ratio and incubated with HUVEC cells grown on microscope cover slips in a six well plate, for three hours in serum free medium. After the three-hour incubation, cells were washed with serum free medium and were allowed to grow in the presence of growth medium for another 20 hours. These cells were then washed with PBS, fixed with 4% paraformaldehyde for 15 minutes and mounted on a hanging drop microscope slide that contain PBS in the well, with the cells facing the well and in contact with PBS. The slides were observed under a Laser Scanning Confocal Microscope (MRC 1000, Bio-Rad) using a 60X oil immersion objective. An Ar/Kr laser light source in combination with the optical filter settings for Rhodamine excitation and emission were used for acquisition of the fluorescence images.

Transfection efficiency of PEI and PEI-PEG complexes was studied using a plasmid DNA pCI-Luc containing Luciferase reporter gene, regulated by CMV promoter. Cells (BL6) were plated at 20000 cells/well in 96 well plates and allowed to grow to 80-90% confluency. They were then incubated with PEI or PEI-PEG/DNA complexes prepared at a charge ratio of 5 (+/−) and a DNA dose of 0.5 μg DNA per well, for 3 hours in serum free medium at 37° C. Cells were allowed to grow in the growth medium for another 20 hours before assaying for the luciferase activity. Luciferase activity in terms of relative light units was assayed using the commercially available kit (Promega) and read on a luminometer, using a 96 well format.

### Example 2

### PEI-PEG-RGD Conjugates and Effect of Ligand on DNA Complexes

RGD peptide with sequence, ACR GDM FGC A, cyclized through the Cys sidechains and purified to >90% by reverse phase HPLC (C 18 column) was obtained from Genemed Synthesis, S. San Francisco. 16.8 mg of the RGD peptide was dissolved in 11 mM HEPES buffer at pH 8.0. To this solution, 41 mg of VS-PEG3400-NHS (Shearwater Polymers) dissolved in dry DMSO (100 μl) was added slowly (over 30 minutes) with stirring using a syringe pump. The reaction mixture was kept stirring at room temperature for another 7 hours. 5 mg of PEI solution after adjusting the pH to 8.0 was added to the above reaction mixture. The reaction mixture was adjusted to pH 9.5 and stirred at room temperature for 4 days. At the end of the reaction, the reaction mixture was lyophilized. The sample was redissolved in 5 mM HEPES at pH 7.0 containing 150 mM NaCl and passed through a G-50 gel filtration column using an elution buffer containing 5 mM HEPES and 150 mM NaCl. Void volume fraction was dialyzed extensively against 5 mM HEPES containing 150 mM NaCl using 25,000 MWCO dialysis tubing. The sample was desalted later by dialyzing against water using a 3500 MWCO membrane. The amount of peptide in the conjugate was determined by estimating the sulfhydryl concentration from Cys side chains. A small fraction of the conjugate was treated with 20 mM DTT to reduce the peptide disulfide bond. This sample was then dialyzed against 0.1M acetic acid containing 1 mM EDTA using a 25000 MWCO dialysis tube, in order to remove excess DTT.

After extensive dialysis, the sulfhydryl concentration was determined using Ellman's reagent and the amine concentration due to PEI was determined using a TNBS assay for primary amines. Based on these assays, peptide conjugation to the PEI was estimated to be 10%. The ability of PEI-PEG-RGD2C to complex with DNA was verified by gel electrophoresis experiments. Complexes formed at or above a charge ratio of 1 failed to migrate into the gel, indicating complete charge neutralization of DNA due to binding of the conjugate.

In order to facilitate the uptake of DNA/polycation complexes, DNA can be condensed into small particles that can be endocytosed by cells. The ability of PEI-PEG-RGD2C to condense DNA into small particles was studied by particle size measurements. Table 1 below shows the particle size of DNA/PEI-PEG-RGD2C at various charge ratios. Table 1 also shows the zeta potential values of DNA/PEI-PEG-RGD2C complexes at various charge ratios. Zeta potential remains low at these charge ratios indicating the formation of a steric coat that masks the surface charge of the complex.

TABLE 1

| Charge | Particle | | | |
|---|---|---|---|---|
| ratio | size (nM) | Std. deviation | Zeta potential | Std. deviation |
| 1.0:1 | 405.6 | 186.6 | −13.3 | 3.65 |
| 1.2:1 | 579.1 | 267.5 | −4.92 | 2.27 |
| 2.0:1 | 58.1 | 24.8 | 6.89 | 6.67 |
| 4.0:1 | 34.9 | 14.8 | 8.98 | 7.81 |
| 10.0:1 | 23.3 | 10.5 | 9.72 | 10.5 |

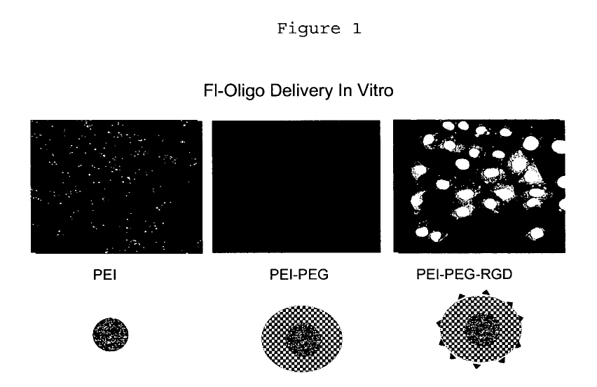

The ability of PEI-PEG-RGD2C to deliver nucleic acids to cells was studied using confocal microscopy using fluorescently labeled oligonucleotide. Confocal microscopy experiments were carried out as described earlier with PEI-PEG. FIG. 1 shows increased cellular uptake of Rh-labeled oligonucleotides complexes in HELA cells at charge ratio 6 with addition of the peptide ligand (RGD) to the distal end of the PEG-Conjugate. The figure shows the delivery of fluorescently labeled oligonucleotide by PEI, PEI-PEG or PEI-PEG-RGD2C to Hela cells.

Fluorescent oligos delivered as a PEI/oligo complex are distributed in the cytoplasm in a punctate pattern indicating vesicular entrapment. With PEI-PEG, the uptake is considerably reduced demonstrating the presence of a steric barrier on the particle and the utility of this steric layer to reduce the nonspecific interactions. When oligo is delivered using PEI-PEG-RGD, there is a considerable increase in the amount of oligo internalized by cells. More importantly, oligo is localized in the nucleus indicating an efficient cytoplasmic delivery by this molecule. The difference in the distribution pattern may indicate different uptake pathways, one that leads to efficient cytoplasmic delivery in the case of PEI-PEG-RGD.

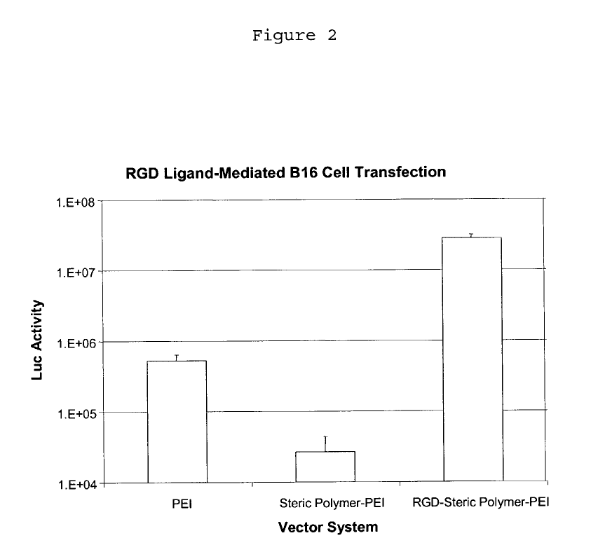

FIG. 2 shows the luciferase activity in cells transfected with of PEI, PEI-PEG or PEI-PEG-RGD and a luciferase plasmid with CMV promoter. Cells transfected with PEI shows high luciferase activity whereas the presence of PEG on the surface of the complex reduces the activity, presumably due to decreased binding. When a PEI-PEG-RGD construct is used for transfection, luciferase activity is restored and even enhanced compared to PEI, This likely indicates a ligand mediated uptake in the case of PEI-PEG-RGD.

### Example 3

### Complexes of Synthetic Vector Reagents with Nucleic Acid

An important hurdle largely neglected in the field is characterization of the colloids formed by the condensing agent and nucleic acid. A good understanding of the nature of the colloids formed is lacking. We have developed formulations and processes to form complexes using physical characterization of the colloids. Our processes have been developed using plasmids (up to 1 mg DNA). Homogeneity of the colloidal complexes is determined using light scattering, zeta potential, and microscopy. The impact of improved homogeneity can be observed from in vivo expression and toxicity. A process has been developed which is scalable, operator independent, and optimized to prepare homogenous 100 nm particles using a flow-through static mixer. This size goal was chosen for two reasons. First, 100 nm average size particles have the best tumor targeting (based on liposome studies). Second, 100 nm average size particles can be sterile filtered in a terminal process step. This eliminates the need to build an aseptic manufacturing plant. A process to separate the product particles from excess, unreacted components has also been developed. The excess reagents present in simple mixing procedures contribute toxicity and instability.

Use of static mixers has been shown to permit formation of homogenous complexes. In studies with small scale mixers, the complexes produced have been shown to have narrower size distribution and smaller average size. In this continuous preparation process, streams of aqueous DNA and of the conjugate is fed into an HPLC static mixer which includes three 50 μl cartridges in tandem and the complexes collected from the output of the final mixer. In the making of each preparation of particles, each stream is fed into the mixer at the same flow rate, and flow rate maintained as the resulting combined stream of DNA and polymer flows through the cartridges. Flow rates can be varied from 250 μl/min. to 5,000 μl/min. Dialysis can be used to remove excess reagents after complexation. The particle size and distribution of size for each preparation made are determined. The results show that particle size can be adjusted by controlling one or more of the parameters including changing the size of the mixing cartridges, the flow rate, the concentration and ratio of the components, and the components of the aqueous phase.

### Example 4:

### Isolation of Genome with Terminal Protein from Adeno Virus

1.5 ml of 8M Guanidine hydrochloride containing 2 mM PMSF are added to 9.3×10¹¹ particles of Av3Luc in 1.5 ml storage buffer containing 15% glycerol and are be kept at room temperature for 15 minutes. The denatured viral sample is transferred to 1,000,000 MWCO dialysis tubing and dialyzed against 4M guanidine hydrochloride containing 1 mM PMSF, at 4° C. Since PMSF has a short half-life in the dialysis conditions used, concentration of PMSF in the dialysis buffer is maintained at 0.5 to 1.0 mM level by the addition of PMSF solution at half-hour intervals. Dialysis is continued with stepwise decrease in the guanidine hydrochloride concentration i.e. 4M, 2M, 1M, with 3 buffer changes for each guanidine hydrochloride concentration. Final dialysis is carried out against TE buffer with no PMSF. Absorption spectrum of the sample obtained is examined for the 260/280 ratio. Viral genome without TP is obtained by treatment of aliquots (0.9 μg) of the DNA with proteinase K (15 μl of 14mg/ml solution) for 48 hours at 56° C.

### Example 5

### Complexes of Synthetic Vector Reagents with Viral Genomic Nucleic Acid and Expression of Encoded Sequences

Transfection complexes of viral genome with cationic liposomes are prepared in 5 mM HEPES buffer at pH 7.0 using equal volume mixing technique. 0.5 ug of the viral genome will be diluted in 10 ul of HEPES buffer. Required amounts of cationic liposomes containing neutral lipids (eg. DOTAP:DOPE (1:1)) from their stock solutions are diluted to 10 ul in HEPES buffer in order to make DNA/liposome complexes with varying charge ratios. DNA and liposome solutions are mixed by adding DNA solution into the liposome solution followed by vortexing for 30 seconds.

### Example 6

### Delivery of Plasmid and Viral Genomic Nucleic Acid by Applied Electric Field

10 ug of plasmid DNA or 10 ug of isolated adenoviral genome encoded for the production of a reporter or therapeutic protein (eg. Luciferase or GMCSF) regulated by a viral or cellular promoter are delivered into the tumor tissue by injection or other physical delivery techniques (eg. Gene-gun). Tissue and cells in the area of delivery are subjected to pulses of electric field in order to distribute the delivered nucleic acid into the cell and into the nucleus of the cell in order to enable the expression of the encoded protein. Application of the electric field is carried out using electrodes designed for easy access to the tissue of interest. For example, needle electrodes for reaching the interior of the tissue and plate electrodes for applying electric field on the surface of the tissue. Electric pulses of different duration and voltage are generated using an electroporator ECM 380 (BTX, San Diego). Biochemical as well as imaging assays are carried out to evaluate the gene delivery and expression in the tissue. In case of secreted proteins, the blood level of the protein is determined using biochemical assays.

### Example 7

### Delivery of RNA to Inhibit Protein Expression: Luciferase Reporter Gene Silencing in Xenografted Tumors Mediated by Co-Transfected dsRNA

To investigate whether interfering RNAs inhibit gene expression in mouse tumor model, we used direct intratumoral injection followed by electroporation to co-deliver naked dsRNA and Luciferase expression plasmid DNA into human MDA-MB-435 tumor xenografted in nude mice. Briefly, a 700 bp DNA fragment derived from firefly Luciferase gene was PCR amplified and a T7 promoter sequence was added to both ends of the DNA fragment during the PCR reaction. The DNA fragment was then used as DNA template for in vitro transcription. In vitro transcription was carried out using an dsRNA generation kit from New England BioLab following its procedure. Two μg of luciferase expression plasmid, pCILuc, was mixed with 0.5, 2, and 5 μg dsRNA derived from Luciferase gene or LacZ gene in a final volume of 30 μl physiologic saline. The DNA/dsRNA mixture in saline solution was directly injected into human MDA-MB-435 tumor xenografted in Ncr Nu/Nu mice with a precision injector (Stepper, Tridake).

Immediately after injection, a procedure of pulsed electrical field was carried out (FIG. 1). A thin layer of conductive gel (KY Jelly) was applied to tumor surface to ensure good contact between the plate electrodes and tumor, and electric pulses were delivered through two external plate electrodes placed at each sides of tumor using an electroporator (BTX ECM 830, San Diego). The parameters for electroporation were as follows: voltage to electrode distance ratio (Electric-Field Strength) was 200-V/cm; duration of each pulse was 20 ms; Interval time between two pulses was 1 second (1 Hz). The number of pulses was 6. Twenty-four hours post DNA injection, tumors were excised after the animals being sacrificed. Each tumor was homogenized in 800 μl of 1× lysis buffer (Promega) in a homogenizing tube (Lysing Matrix D, Q-BIOgene) using a Fastprep (Q-BIOgene) with speed at 4 for 40 seconds at 4° C. The homogenates were centrifuged at 14,000 rpm for 2 minutes after incubation on ice for 30 minutes. The supernatant was transferred into a fresh tube and 10 μl was used for luciferase activity assay using the Luciferase assay kit (Promega) and a Luminometer (Monolight 2010, Analytic Luminescence Lab).

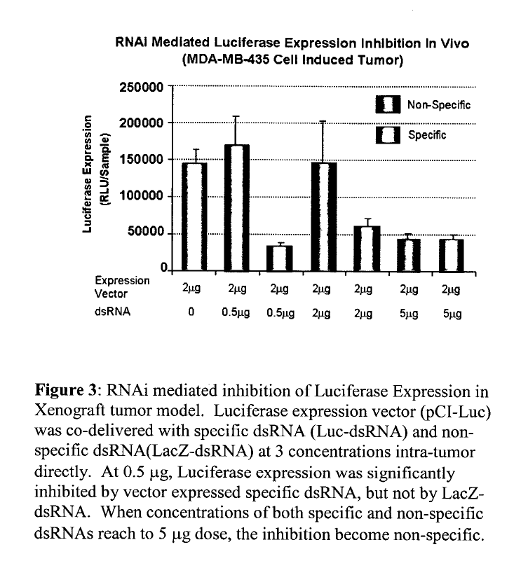

As illustrated in FIG. 3, the co-delivered dsRNA derived from Luciferase gene was able to silence Luciferase expression in xenografted tumor. As low as 0.5 μg dsRNA was enough to achieve significant gene silencing against 2 μg of co-delivered pCILuc plasmid DNA. Non-specific dsRNA interference effect was observed when 5 μg dsRNA derived from LacZ gene was co-delivered with 2 ug of pCILuc plasmid DNA. No non-specific effect was observed at lower doses of dsRNA (0.5 μg and 2 μg). To the best of our knowledge, this is the first that dsRNA mediated specific gene silencing was observed in xenografted tumor in adult mice.

What is claimed is:

1. A method of treating or alleviating the symptoms of a disease in a mammal, comprising administering a therapeutically effective amount of a nucleic acid composition to a tissue of said mammal, wherein said nucleic acid is comprised within a nucleic acid encoding a viral genomic sequence.

2. The method according to claim 1, wherein said viral genomic sequence is capable of repeated self-replication in vivo.

3. The method according to claim 1, wherein said nucleic acid is comprised within a synthetic vector.

4. The method according to claim 1, wherein a pulsed electric field is applied substantially contemporaneously to said tissue together with said nucleic acid composition.

5. The method according to claim 1, wherein said nucleic acid composition reduces or increases the expression of a protein or polypeptide in said mammal.

6. The method according to claim 5, wherein said nucleic acid composition decreases the expression of a protein or polypeptide selected from the group consisting of an oncogene, a protein kinase and a transcription factor.

7. The method according to claim 5, wherein said nucleic acid composition increases the expression of a protein or polypeptide selected from the group consisting of a tumor suppressor protein, an immunostimulatory cytokine and an oncolytic protein.

8. The method according to claim 7, wherein said protein or polypeptide is an immunostimulatory cytokine selected from the group consisting of GM-CSF, IL-1, IL-12, IL-15, an interferon, B-40, B-7, and tumor necrosis factor.

9. A method of treating or alleviating the symptoms of a disease in a mammal, comprising administering a therapeutically effective amount of a nucleic acid composition to a tissue of said mammal, wherein said nucleic acid is a single or double stranded oligonucleotide and wherein said nucleic acid is either (i) comprised within a synthetic vector, or (ii) applied substantially contemporaneously with a pulsed electric field to said tissue.

10. The method according to claim 9, wherein said nucleic acid composition reduces the expression of a protein or polypeptide in said mammal.

11. The method according to claim 10, wherein said nucleic acid composition is selected from the group consisting of an antisense oligonucleotide, RNAi, and a non-naturally occurring oligonucleotide.

12. The method according to claim 10, wherein said protein or polypeptide is selected from the group consisting of an oncogene, a protein kinase and a transcription factor.

13. The method according to claim 12, wherein said protein or polypeptide is selected from the group consisting of BCL2, VEGF R2, NF kappa B, RAF kinase, PKC delta, HER2, and bFGF.

14. A method of treating or alleviating the symptoms of a disease in a mammal, comprising applying a therapeutically effective amount of an anti-inflammatory composition into a joint of said mammal and substantially contemporaneously applying a pulsed electric field to said joint.

15. The method according to claim 14, wherein said anti-inflammatory composition comprises a compound selected from the group consisting of a nucleic acid, a small molecule drug, a peptide, and a protein.

16. The method according to claim 15, wherein said anti-inflammtory composition is a nucleic acid selected from the group consisting of DNA, RNA, a viral genome lacking a capsid protein, a synthetic non naturally occurring nucleic acid, and an oligonucleotide.

17. The method according to claim 16, wherein said nucleic acid is a DNA, RNA, or viral genome encoding at least one anti-inflammatory protein.

18. The method according to claim 15, wherein said anti-inflammtory composition is a single or double stranded oligonucleotide that decreases expression of a pro-inflammatory cytokine in said joint.

19. The method according to claim 18, wherein said oligonucleotide is a non-naturally occurring oligonucleotide.

20. The method according to claim 18, wherein said oligonucleotide is a short interfering RNA or an interfering double stranded RNA.
